(19) United States
(12) Patent Application Publication
(10) Pub. No.: US 2026/0355533 A1
(43) Pub. Date: Nov. 12, 2026

(54) FACEMASK

(71) Applicants: GUANGDONG NEWDERMO BIOMEDICAL TECH CO LTD, Guangdong (CN); SILKN HOLDING B.V., Rotterdam (NL)

(72) Inventors: Cai QINGYAO, Shiji Town (CN); Oscar MIDDENDORP, Rotterdam (NL)

(21) Appl. No.: 19/646,007

(22) Filed: Apr. 13, 2026

Related U.S. Application Data
(63) Continuation of application No. PCT/NL2024/050632, filed on Nov. 25, 2024.

(30) Foreign Application Priority Data
Oct. 13, 2023 (CN) 202311330494.5
Nov. 23, 2023 (CN) 202311574900.2
Nov. 23, 2023 (CN) 202323172721.4

(51) Int. Cl. A61N 5/06

(52) U.S. Cl. CPC A61N 5/0616; A61N 2005/0632; A61N 2005/0647; A61N 2005/0652; A61N 2005/0654; A61N 2005/0664

(57) ABSTRACT

The present invention relates to a facemask such as a phototherapy mask for the face, comprising a phototherapy feature or function for placement at the face, the facemask comprising:—a mask body, at which a plurality of lamp beads (100) for irradiation of light are arranged,—a plurality of protruding members that are provided at one side relative to the lamp bead to provide a distance or a minimum distance between the lamp bead and a face of the person.

The phototherapy mask generally includes a mask, a flexible circuit board, and a face contact member which are stacked in sequence; the light beads are arranged on the flexible circuit board and are controlled by the flexible circuit board; the face contact member is used to abut against the human face skin, that is, to isolate the human face skin and the flexible circuit board to ensure the wearing comfort of the phototherapy mask;

However, most of the phototherapy masks available currently focus on compact, flat and thin structural designs. Although this may improve their appearance and portability it provides direct transfer from the light beads to the skin.

The present invention is based on the realization of the inventor that such direct transfer from the light beads to the skin provides disadvantages to be solved, such as part of the skin that are irradiated with too much intensity in part of the skin that are irradiated with too low intensity. It is therefore a goal of the present invention to provide a solution to the state of the art that allows for balanced irradiation of the skin in relation to requirements of the skin to be irradiated.

To this end, the present invention provides a facemask, such as a phototherapy mask for the face, comprising a phototherapy feature or function for placement at the face, the facemask comprising:
a mask body, at which a plurality of lamp beads (100) for irradiation of light are arranged,
a plurality of protruding members that are provided at one side relative to the lamp bead to provide a distance or a minimum distance between the lamp bead and a face of the person.

It is an advantage of such a facemask according to the present invention to provide a facemask, such as a phototherapy mask, that may increase the light guide spacing so as to obtain a light spot of a target size and improve the phototherapy effect. It is a further advantage of such a facemask or an embodiment thereof that respective predetermined areas of the face may be provided with a respective predetermined amount of irradiation for optimal results of the face by the irradiation. It is a further advantage of such a facemask or an embodiment thereof that the irradiation may be guided towards the skin over set distance. It is a further advantage of such a facemask according to the present invention or an embodiment thereof that it allows for focusing of light to provide higher intensity at the irradiated area or to allow for focusing the flights to provide lower intensity at the irradiated area. That is preferably achieved by converging lens structures or diverging the instructors to be comprised in the protruding members.

Furthermore, it is an advantage of such a facemask or an embodiment thereof that part of the facemask may during use contact the face or that part of the facemask may during use be at a distance relative to the face while still maintaining optimal irradiation of such relevant part of the face. This means that it is advantageously achieved that single size facemask or facemask in a limited number of sizes may provide satisfactory treatment of the face.

Also, materials may be used to provide advantageous manufacturing advantages, such as relatively stiff materials with better durability as contact with the face overall surface of the mask is not a requirement with the application of the present invention or embodiments thereof.

According to a first preferred embodiment according to the present invention, the protruding member comprises a light-transmitting structure. An advantage of such embodiment is that the structure may be in between the face and the light bead.

According to a further preferred embodiment according to the present invention, a respective protrusion corresponds with the position of a respective lamp bead. An advantage of such embodiment is that the device providing the distance may also be used as a light guide between the light bead and the face.

According to a further preferred embodiment, a respective protrusion (210) comprises a lens structure that is arranged for converging or diverging of the light, and/or is used to change the size of an irradiated light spot at the skin. Such an embodiment advantageously provides a predetermined irradiation intensity at a perspective predetermined area of the skin.

According to a further preferred embodiment, a groove is provided on one side of the protrusion, preferably at the sides thereof directed towards the lamp bead.

According to a further preferred embodiment, the facemask comprises a flexible circuit board, preferably wherein the mask body comprises the flexible circuit board.

According to a further preferred embodiment, the facemask comprises a cover member, preferably wherein the mask body comprises the cover member.

According to a further preferred embodiment, the facemask comprises a face contact member (200), preferably wherein the mask body comprises the face contact member.

According to a further preferred embodiment, at least one of the plurality of protrusion members (210) is arranged at the face contact member (200) or wherein the plurality of protruding members are arranged as the face contact member.

According to a further preferred embodiment, the facemask is embodied as follows:
the face contact member (200) comprises a first panel (220),
a respective protrusion member (210) is arranged on the first panel (220), and
the respective protrusion member (210) protrudes outward in a direction away from the lamp bead (100) and protrudes out of the wall surface of the first panel (220) away from the mask body.

According to a further preferred embodiment, in the facemask:
the protruding member (210) and the face contact member or first panel (220) are an integral structure.

According to a further preferred embodiment, in the facemask:
the face contact member or the first panel (220) is provided with a plurality of through holes (230) corresponding to respective lamp beads (100), and the protruding members (210) are installed at the through holes (230).

According to a further preferred embodiment, in the facemask:
the face contact member (200) comprises a first panel (220) and a second panel (240),
the protruding member (210) is arranged on the first panel (220), and the second panel (240) is arranged at the wall surface of the first panel (220) adjacent to the mask body and covers a respective lamp bead (100).

According to a further preferred embodiment, in the facemask:
the face contact member (200) comprises a first panel (220) and a second panel (240),
the protruding member (210) is arranged at the first panel (220), and the second panel (240) is arranged at a side of the first panel (220) away from the mask body and covers the protruding member (210).

According to a further preferred embodiment, the lens structure comprises lens means with a meniscus lens structure, preferably wherein at least one of the lens structures comprises a converging meniscus lens or lens part and/or wherein at least one of the lens structures comprises a diverging meniscus lens or lens part.

According to a further preferred embodiment, in the facemask the mask body is arranged the body adapts to the shape of the face and wherein each protruding member is adapted to provide a focusing adjustment in relation to the coverage of the respective skin.

Further advantages, features and details of the present invention will be further elucidated on the basis of a description of one or more preferred embodiments with reference to the accompanying figures. Similar yet not necessarily identical parts of different preferred embodiments may be indicated with the same reference numerals.

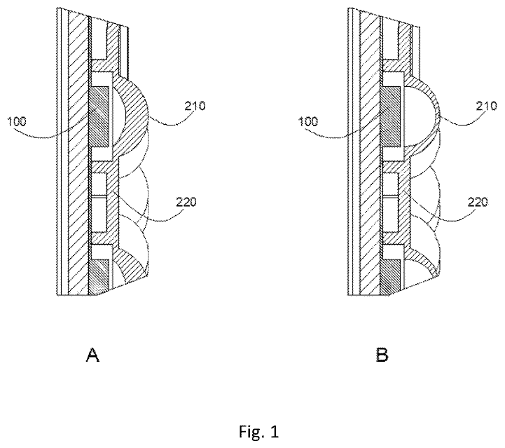

FIG. 1 is a schematic diagram of two lens structures formed by protrusions according to a preferred embodiment according to the present invention.

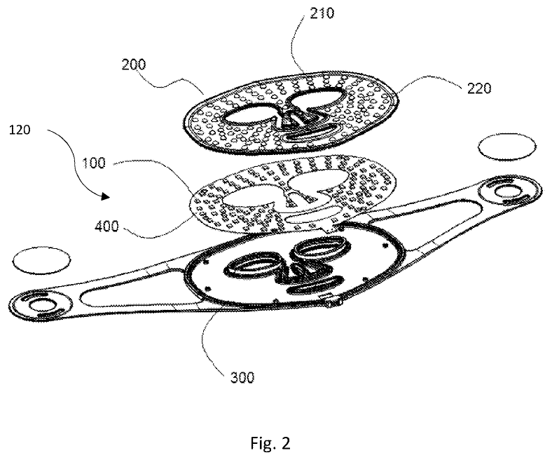

FIG. 2 is a schematic diagram of a three-dimensional unfolded structure of a phototherapy mask provided in solution according to a preferred embodiment according to the present invention.

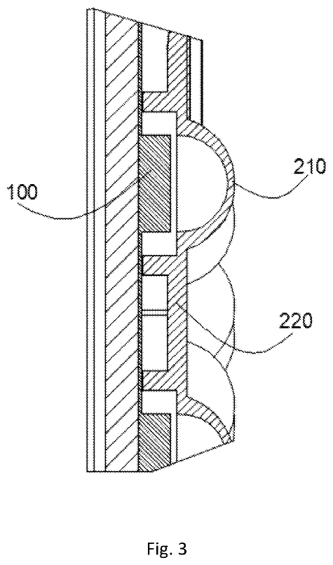

FIG. 3 is a cross-sectional view of a phototherapy mask provided in solution a according to a preferred embodiment according to the present invention;

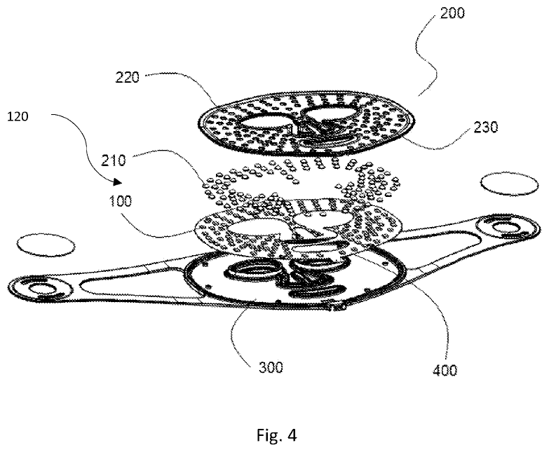

FIG. 4 is a schematic diagram of a three-dimensional unfolded structure of a phototherapy mask provided in solution b according to a preferred embodiment according to the present invention.

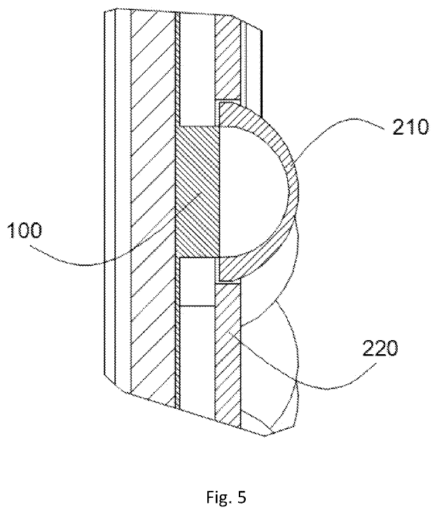

FIG. 5 is a cross-sectional view of a phototherapy mask provided in solution b according to a preferred embodiment according to the present invention.

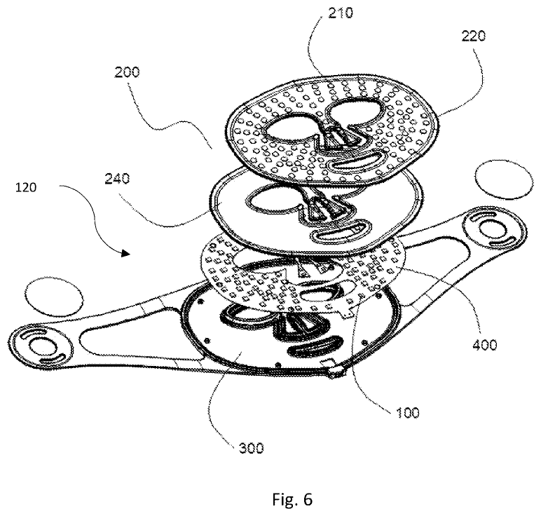

FIG. 6 is a schematic diagram of a three-dimensional unfolded structure of a phototherapy mask according to a further or second preferred embodiment according to the present invention.

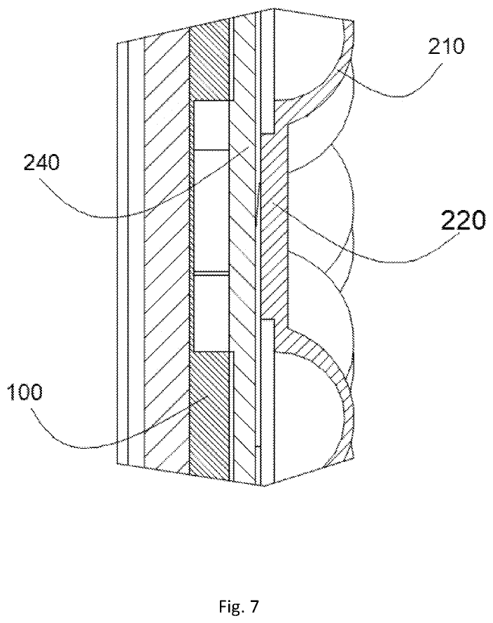

FIG. 7 is a cross-sectional view of a phototherapy mask according to a further or second preferred embodiment according to the present invention.

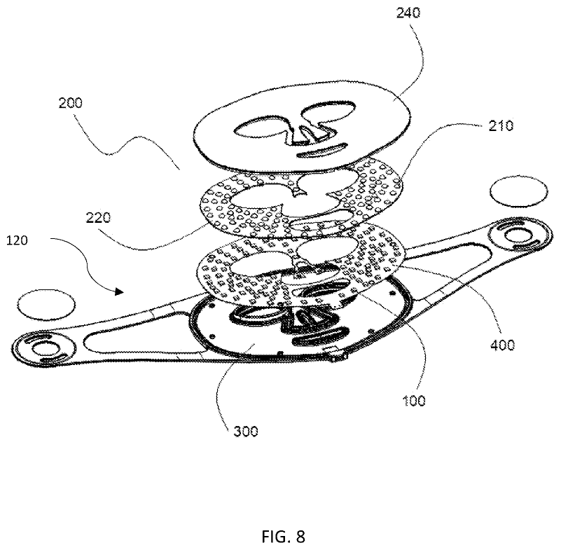

FIG. 8 is a schematic diagram of a three-dimensional unfolded structure of a phototherapy mask according to a further or third preferred embodiment according to the present invention.

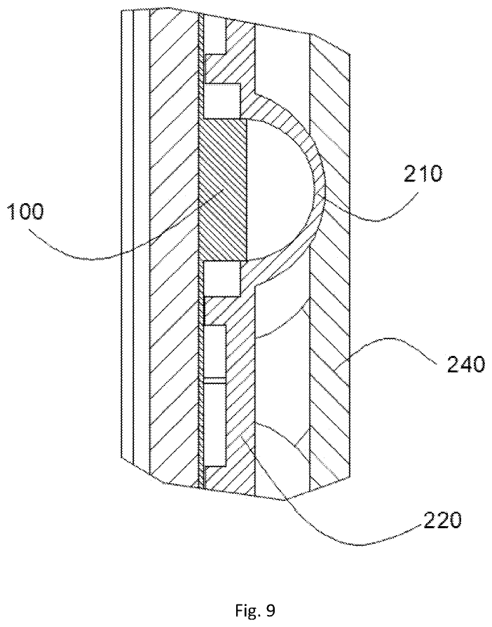

FIG. 9 is a cross-sectional view of a light therapy mask according to a further or third preferred embodiment according to the present invention.

A technical solution of preferred embodiments according to the present invention is further described below with reference to the accompanying drawings and through preferred, nonlimiting, implementation methods.

As shown in FIGS. 1 to 9, a specific embodiment of the present invention discloses a phototherapy mask, including a mask body 120, comprising a plurality of lamp beads 100. A plurality of protruding members 210 are disposed on one side of the lamp beads 100 for providing a predetermined distance, or increasing, the distance between the lamp beads 100 and a human face. Alternatively, a distance between light guides is extended by increasing the distance to the human face, providing conditions for implementing scattered light or focused light, thereby improving the phototherapy effect.

Preferably, the protrusion 210 is a light-transmitting structure. The light-transmitting structure provides support and provides the light-guiding spacing without affecting the normal transmission of light from the lamp beads, thereby achieving a predetermined and/or desired phototherapy effect.

Furthermore, the position of a protrusion 210 corresponds to a respective lamp bead 100, and the protrusion corresponding to the lamp bead may advantageously guide light and provide the basic conditions for focusing or diffusing light.

Further preferably, the protrusion 210 is a lens structure, and the size of the irradiated light spot on the face or the skin thereof may be defined on the basis of determining spacing of the light guide. Several light guide effects may be achieved according to the shape of the protrusion to meet preferred needs.

For example, when the protrusion is a convex lens structure, it may achieve an effective light focusing effect, which is used to achieve the effect of local higher intensity irradiation phototherapy.

With this, the phototherapy effect on the sides of the nose and the corners of the eyes are preferably achieved.

When the protrusion is a concave lens structure, it may achieve a diverging effect or otherwise an astigmatism effect, and the light spots overlap with each other, preventing an area to be missed and/or achieving a more comprehensive coverage. With this, an effective the phototherapy effect is advantageously achieved.

Alternatively, when both end surfaces of the protrusion are planar structures, the protrusion itself does not have the effect of diverging, astigmatism or focusing, and only has the effect of increasing the spacing. According to a law of light irradiation, a larger light spot may also be formed on the face, which has a similar effect to when the protrusion is a concave lens structure, reducing the missing area, expanding the irradiation coverage, and improving the phototherapy effect. Alternatively, when the protrusion plays the function of astigmatism, there is no need to expand the light spot coverage by densely arranging lamp beads, which means that the use of lamp beads may be reduced, and the production cost of the mask may be reduced.

It is mentioned that based on structures of the above-mentioned several lenses, and according to the illumination requirements of the actual layout, the lens type and structure are preferably predetermined so that the illumination angle formed by the light transmitting through the protrusions of the lamp beads is between 10° and 170°, thereby achieving targeted enhancement of the focusing/scattering coverage according to different parts, and adjusting the design to better adapt to the human face to ensure the phototherapy effect.

Further preferably, a groove is provided on the side of the protruding member 210 close to the lamp bead 100, and the wall surface of the groove away from the lamp bead is an arc surface. The design of the groove may provide a receiving position, which may facilitate the protruding member cover to be arranged on the outside of the lamp bead, so that the overall structure may be more compact and prevent the protruding member from directly contacting the lamp bead.

Furthermore, it is mentioned that, on this basis, if the protruding member is preferred to have a focusing effect, as shown in Figure A in FIG. 1, the depth of the groove is preferably reduced, and the wall surface of the protruding member away from the lamp bead is in an arc shape, or at least the edge portion needs to be cut, so that the protruding member as a whole provides an effect of being thick in the middle and thin on both sides, maintaining the structure of the convex lens and achieving the focusing effect;

If the raised part is preferred to have a diffuse light effect, as shown in FIG. 1B, the wall away from the lamp bead includes but is not limited to a flat structure/arc surface, but it is preferably provided that the raised part as a whole has a thin middle and thick edge structure, maintain the structure of the concave lens, and thereby achieve the diffuse light effect.

Further preferably, the facemask includes a face contact member 200, a protruding member 210 is arranged on the face contact member 200, and the face contact member 200 is installed on the mask body 100. The mask body includes a cover member 300 and a flexible circuit board 400. The flexible circuit board is closely arranged on the cover member. The lamp bead 100 is protruded and arranged on the wall of the flexible circuit board 400 away from the cover member 300. The edge of the face contact member 200 is closely arranged on the cover member 300, and is detachably fastened by means of snap connection, bonding or magnetic connection, the face contact member is covered on the flexible circuit board, and the light of the lamp bead penetrates the face contact member to irradiate outward.

Furthermore, as a device for mask use, considering the layout and compactness of the overall structure, the lamp bead used is a flat and thin lamp bead body, preferably an LED lamp bead. Side wings extending outward are provided on both sides of the cover member, and Velcro is provided at the end of the side wings, and the two side wings are connected by Velcro to achieve a wearing effect about the head of the face, thereby forming a complete light therapy mask structure for actual application. Other common structural components such as electrical components and remote control components and circuit connection methods are not described in detail.

### First Preferred Embodiment

In a first preferred embodiment, the face contact member 200 includes a first panel 220, and a protrusion 210 is disposed on the first panel 220. The protrusion 210 protrudes outward in a direction away from the lamp bead 100 and protrudes out of the wall of the first panel 220 away from the mask body.

The face contact member preferably has a single-layer structure, which may effectively ensure the light transmittance, so that the light may be advantageously transmitted outward. By preferably setting a protrusion protruding in the direction away from the mask body, the effect of increasing the light guide spacing is achieved, and the required focusing or diffusing effect is also achieved.

As shown in FIG. 2 and FIG. 3, in scheme A of the present embodiment, the protrusion 210 and the first panel 220 are an integral structure. The protrusion and the first panel are an integral structure, which ensures the overall continuity and sufficient structural strength, and the overall light transmittance is relatively uniform. It is also convenient for processing and production, and the protrusion and the first panel are an integral structure and are manufactured by an integrated molding method, such as one-time casting molding, two-time casting molding, etc.

Since the protrusion and the first panel are the parts that contact the human face, they may be made of flexible material, preferably a silicone; and the design of the groove not only serves the purpose of accommodating the parts, but also increases the extrusion deformable spacing of the protrusion, achieving soft contact with the human face, thereby providing an advantageous contact.

As shown in FIG. 4 and FIG. 5, in the scheme B of the present embodiment, the first panel 220 is provided with a plurality of through holes 230 corresponding to the lamp beads 100, and the protrusions 210 are installed at the through holes 230. Compared with the scheme A of the embodiment 1, a similar single-layer structure may be used, and the light guide spacing may be increased by the protrusions protruding outward, and the desired focusing or diffusing effect may also be achieved;

A difference is that the assembled structure adopted in this embodiment may be matched by clamping or bonding, and protrusions with different light transmittances may be selected according to different bone parts or facial acupuncture points to meet different irradiation intensities;

In addition, protrusions of different strengths may be selected according to different parts to achieve a combination of local strong support and local soft support, so that the mask may fit the face better and more adaptably; because it is a dispersed and independent protrusion structure, even if a harder structure is used, it will prevent excessively damage the deformation effect of the entire face-contacting part, and a normal bending and use of the mask is achieved.

### Second Embodiment

As shown in FIG. 6 and FIG. 7, in this embodiment, the face contact member 200 includes a first panel 220 and a second panel 240. The protrusion 210 is disposed on the first panel 220. The second panel 240 is disposed on the wall of the first panel 220 adjacent to the mask body, covering the lamp bead 100.

Compared with the scheme A of the embodiment 1, it is similar that the light guide spacing may be increased by the outwardly protruding protrusions, and the required light focusing or light diffusion effect may be achieved; and the protrusions and the first panel are an integrated structure of silicone material;

A difference is that in this embodiment, a second panel is used, and the second panel is also made of a flexible material, preferably a silicone material. The second panel is arranged between the lamp bead and the protruding member, which may further increase the light guide spacing to meet the required effect. Since the flexible circuit board and the lamp bead are electrical components, the added second panel may enhance the insulation effect to a certain extent, play a protective role, and improve the safety of use.

With using the second panel, the protruding part will also have a corresponding effect. The design of the groove may increase the extrusion deformable spacing of the protruding part, and the light-guiding spacing may be shortened when it is deformed by contact with the human face. The addition of the second panel may compensate for the shortened light-guiding spacing caused by the deformation to a certain extent, making its overall effect better than the method of supporting only by the protruding part with a groove in scheme a of embodiment 1. Furthermore, its overall structure is less compact as the scheme of embodiment 1.

### Third Embodiment

As shown in FIG. 8 and FIG. 9, in this preferred embodiment, the face contact member 200 includes a first panel 220 and a second panel 240. The protrusion 210 is disposed on the first panel 220, and the second panel 240 is disposed on a side of the first panel 220 directed away from the mask body and covers the protrusion 210.

Compared with the solution of the above second preferred embodiment, similar points are that the light guide spacing may be increased by the outwardly protruding protrusions, and the desired light focusing or light diffusion effect may be achieved. The protrusions and the first panel are an integrated structure of silicone material. A second panel is also provided, and the second panel is also made of a flexible material, preferably silicone material, which may further increase the light guide spacing and/or to a certain extent compensate for the light guide spacing shortened by the deformation of the protrusions and provide an insulating protection effect.

A difference is that the second panel in this embodiment is arranged on the raised side of the raised member, so that the portion in contact with the face is in a flat state as a whole, instead of a situation where multiple raised points of the raised member contact the face, which may further improve the tactile experience, and the effect may be preferred in relation to that of the above embodiments.

Furthermore, the groove is close to the lamp bead and the cover is arranged outside the lamp bead. Compared with Example 2, the light guide spacing reduces the thickness of the first panel, and the structure is more compact while meeting the illumination range.

It is mentioned that in the above-mentioned multiple embodiments, the first panel, the second panel, and the protrusion are preferably transparent structures or translucent structures to achieve the desired light-guiding effect. The light-guiding spacing range to be achieved is between 2 and 7 mm. The thickness of the panel or the protruding thickness of the protrusion is adjusted according to the combination of different embodiments. The above-mentioned light-guiding spacing range is achieved through coordinated cooperation to achieve the desired focusing and scattering effects.

Furthermore, it is preferred to adapt the size of the protruding members to a respective area of the face it is arranged at. Also the length of the protruding members is thereto adapted. In this sense, the length is the distance between the light bead and the distant limit of the protruding member. At a location of the face where there is a larger distance between light bead and the skin of the face, parameters of the protruding members are adapted, such parameters including the strength of the lens, whether the lens is to be diverging or converging. Also whether the distance between the light bead and the face is to be determined by physical contact between the facemask and the respective area of the skin determines for instance the length and/or other parameters of the respective protruding member.

The present invention is described in the foregoing on the basis of preferred embodiments. Different aspects of different embodiments are expressly considered disclosed in combination with each other and in all combinations that on the basis of this document, when read by a skilled person of the area of skill, fall within the scope of the invention or are deemed to be read with the disclosure of this document. These preferred embodiments are not limitative for the scope of protection of this document. The rights sought are defined in the appended claims.

1. A facemask, such as a phototherapy mask for the face, comprising a phototherapy feature or function for placement at the face, the facemask comprising:
a mask body, at which a plurality of lamp beads for irradiation of light are arranged, and
a plurality of protruding members that are provided at one side relative to the lamp bead to provide a distance or a minimum distance between the lamp bead and a face of the person.

2. A facemask according to claim 1, wherein the protruding member comprises a light-transmitting structure.

3. A facemask according to claim 1, wherein a respective protruding member corresponds with the position of a respective lamp bead.

4. A facemask according to claim 1, wherein a respective protruding member comprises a lens structure that is arranged for converging or diverging of the light, and/or is used to determine the size of an irradiated light spot at the skin.

5. A facemask according to claim 1 wherein a groove is provided on one side of the protrusion, preferably at the sides thereof directed towards the lamp bead.

6. A facemask according to claim 1, further comprising a flexible circuit board, preferably wherein the mask body comprises the flexible circuit board.

7. A facemask according to claim 1, further comprising a cover member, preferably wherein the mask body comprises the cover member.

8. A facemask according to claim 1 comprising a face contact member, preferably wherein the mask body comprises the face contact member.

9. A facemask according to claim 8, wherein at least one of the plurality of protrusion members is arranged at the face contact member or wherein the plurality of protruding members are arranged as the face contact member.

10. A facemask according to claim 8, wherein:
the face contact member comprises a first panel,
a respective protrusion member is arranged on the first panel, and
the respective protrusion member protrudes outward in a direction away from the lamp bead and protrudes out of the wall surface of the first panel away from the mask body.

11. A facemask according to claim 8, wherein:
the protruding member and the face contact member or first panel are an integral structure.

12. A facemask according to claim 8, wherein:
the face contact member or the first panel is provided with a plurality of through holes corresponding to respective lamp beads, and the protruding members are installed at the through holes.

13. A facemask according to claim 8, wherein:
the face contact member comprises a first panel and a second panel,
the protruding member is arranged on the first panel, and the second panel is arranged at the wall surface of the first panel adjacent to the mask body and covers a respective lamp bead.

14. A facemask according to claim 8, wherein:
the face contact member comprises a first panel and a second panel,
the protruding member is arranged at the first panel, and the second panel is arranged at a side of the first panel away from the mask body and covers the protruding member.

15. A facemask according to claim 4, wherein the lens structure comprises lens means with a meniscus lens structure, preferably wherein at least one of the lens structures comprises a converging meniscus lens or lens part and/or wherein at least one of the lens structures comprises a diverging meniscus lens or lens part.

16. A facemask according to claim 1, wherein the mask body is arranged the body adapts to the shape of the face and wherein each protruding member is adapted to provide a focusing adjustment in relation to the coverage of the respective skin.
